(19) United States
(12) Patent Application Publication
(10) Pub. No.: US 2022/0184334 A1
(43) Pub. Date: Jun. 16, 2022

(54) Endotracheal Tube

(71) Applicant: Tien-Sheng Chen, Taipei City (TW)

(72) Inventor: Tien-Sheng Chen, Taipei City (TW)

(21) Appl. No.: 17/540,382

(22) Filed: Dec. 2, 2021

(30) Foreign Application Priority Data
Dec. 16, 2020 (TW) 109144561

(51) Int. Cl. A61M 16/04

(52) U.S. Cl. CPC A61M 16/0488; A61M 16/0418; A61M 16/0475

(57) ABSTRACT

An endotracheal tube has a curved tube body and an oblique cutting surface. The curved tube body has a front end, and the curved tube body has a center axis, an inner axis and an outer axis. The oblique cutting surface has a tip, and the oblique cutting surface is located at the front end and has the following characteristics: the oblique cutting surface has a circular projection surface in the vertical plane and a tip projection point on the circumference of the circular projection surface, the inner axis has an inner axis projection point on the circumference of the circular projection surface, and the tip projection point and the inner axis projection point have an angle θ, where 15°<θ<45°.

### BACKGROUND OF THE INVENTION

### 1. Field of the Invention

The present invention relates to an endotracheal tube; more particularly, the present invention relates to an endotracheal tube having a tip which can be turned to face toward the center of the trachea upon movement of a laryngoscope blade so as to increase the success rate of endotracheal intubation.

### 2. Description of the Related Art

Please refer to FIG. 1 and FIG. 2, which illustrate a conventional endotracheal tube 800 having a conventional endotracheal tube tip 801 commonly used in the current medical profession. When a doctor performs endotracheal intubation on a patient, the conventional endotracheal tube 800 needs to be placed onto a laryngoscope blade 900, and then the laryngoscope blade 900 is inserted into the patient's trachea. The width of such a laryngoscope blade 900 is about 2.2 to 2.4 cm. Ideally, if the doctor wants to ensure that the conventional endotracheal tube 800 placed onto the right side of the laryngoscope blade 900 faces toward the center of the patient's tracheal passage 700, the width of the patient's larynx needs to be greater than 4.4 to 4.8 cm. However, the width of some adults' larynxes is smaller than 4 cm. Therefore, under the condition that the width of the patient's larynx is smaller than 4 cm, it is not feasible for the existing laryngoscope blade 900 to cause the conventional endotracheal tube 800, which has the conventional endotracheal tube tip 801 oblique from the right side to the left side, to face toward the center of the patient's tracheal passage 700. As a result, the failure rate of endotracheal intubation using such a laryngoscope is still high. Also, because the conventional endotracheal tube tip 801 is oblique from the right side to the left side, then if the conventional endotracheal tube tip 801 cannot face toward the center of the patient's tracheal passage 700, the conventional endotracheal tube tip 801 can easily cause soft tissue injury to the larynx when the doctor moves the conventional endotracheal tube 800.

Further, for those patients having a smaller throat diameter, the doctor's operable width of the larynx will be even narrower. Under such circumstances, it is more difficult for the conventional endotracheal tube tip 801 to face toward the center of the patient's trachea, thereby increasing the difficulty of endotracheal intubation. However, because the laryngoscope is a commonly used medical device in the current medical profession, there is a need to improve the design of the conventional endotracheal tube 800 itself so as to increase the success rate of endotracheal intubation using the laryngoscope, as well as to reduce the risk of injury to the soft tissue of the patient's larynx caused by the conventional endotracheal tube tip 801.

Therefore, there is a need to provide an endotracheal tube to mitigate and/or obviate the aforementioned problems.

### SUMMARY OF THE INVENTION

It is an object of the present invention to provide an endotracheal tube capable of increasing the success rate of endotracheal intubation via guidance of an endotracheal tube tip to face toward the center of a tracheal passage upon movement of a laryngoscope blade when the width of a larynx is limited.

To achieve the abovementioned object, the endotracheal tube of the present invention comprises a curved tube body and an oblique cutting surface. The curved tube body has a front end, and the curved tube body has a center axis, an inner axis and an outer axis. The oblique cutting surface has a tip, and the oblique cutting surface is located at the front end. The endotracheal tube has the following characteristics: the oblique cutting surface has a circular projection surface on a vertical plane, the tip has a tip projection point on the circumference of the circular projection surface, the inner axis has an inner axis projection point on the circumference of the circular projection surface, and the tip projection point and the inner axis projection point form an angle θ, where −45°<θ<90°.

Because the cutting direction of the oblique cutting surface located at the front end of the endotracheal tube of the present invention is oblique from a right side tube portion to a left side tube portion, the endotracheal tube tip can therefore face toward the center of a patient's tracheal passage if the width of the patient's larynx is smaller than 4 cm, and this ability will increase the success rate of endotracheal intubation, especially by increasing the success rate of endotracheal intubation on patients having a smaller oral cavity or narrower larynx, which complicate endotracheal intubation, and this design also reduces the risk of soft tissue injury caused by the endotracheal tube tip.

Other objects, advantages, and novel features of the invention will become more apparent from the following detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only and not as a definition of the invention.

In the drawings, wherein similar reference numerals denote similar elements throughout the several views:

FIG. 1 illustrates a schematic drawing of a conventional endotracheal tube placed onto a laryngoscope blade.

FIG. 2 illustrates a schematic drawing showing corresponding positions of the conventional endotracheal tube placed onto the laryngoscope blade after being inserted into a human trachea.

FIG. 3 illustrates a schematic drawing of an endotracheal tube placed onto a laryngoscope blade according to a first embodiment of the present invention.

FIG. 4A illustrates a schematic drawing of the endotracheal tube according to the first embodiment of the present invention.

FIG. 4B illustrates a schematic drawing showing a circular projection surface on a vertical plane relative to an oblique cutting surface of the endotracheal tube according to the present invention.

FIG. 5 illustrates a cross-sectional schematic drawing of a front end of the endotracheal tube according to the first embodiment of the present invention.

FIG. 6 illustrates a schematic drawing showing corresponding positions of the endotracheal tube placed onto the laryngoscope blade after being inserted into a human trachea according to the present invention.

FIG. 7 illustrates a right side view of the endotracheal tube according to a second embodiment of the present invention.

FIG. 8 illustrates a left side view of the endotracheal tube according to the second embodiment of the present invention.

FIG. 9 illustrates a top view of the endotracheal tube according to the second embodiment of the present invention.

FIG. 10 illustrates a bottom view of the endotracheal tube according to the second embodiment of the present invention.

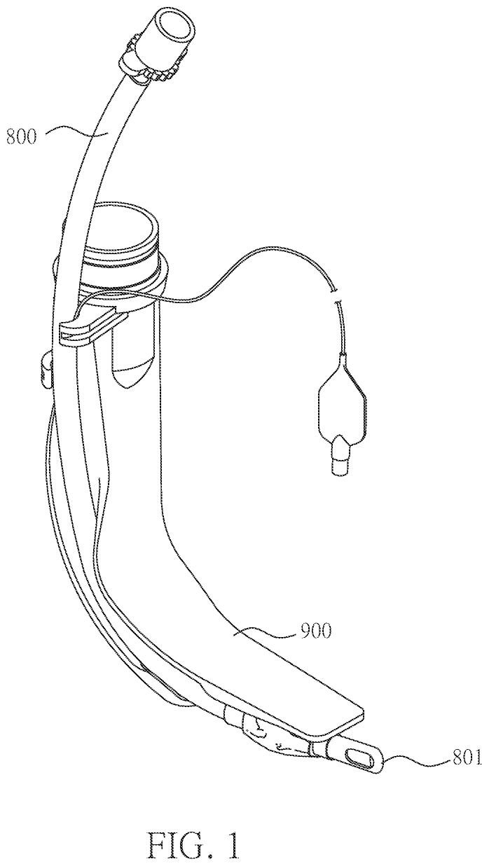

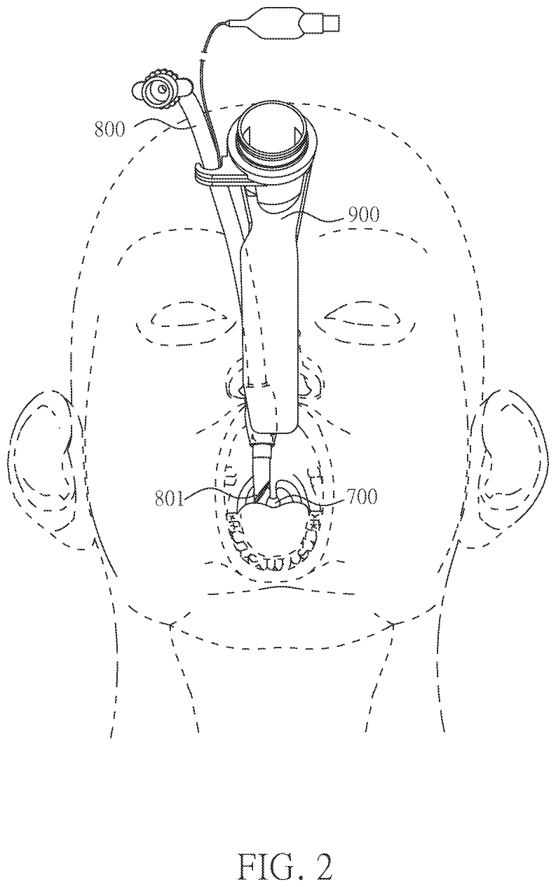

### DETAILED DESCRIPTION OF THE PREFERRED EMBODIMENT

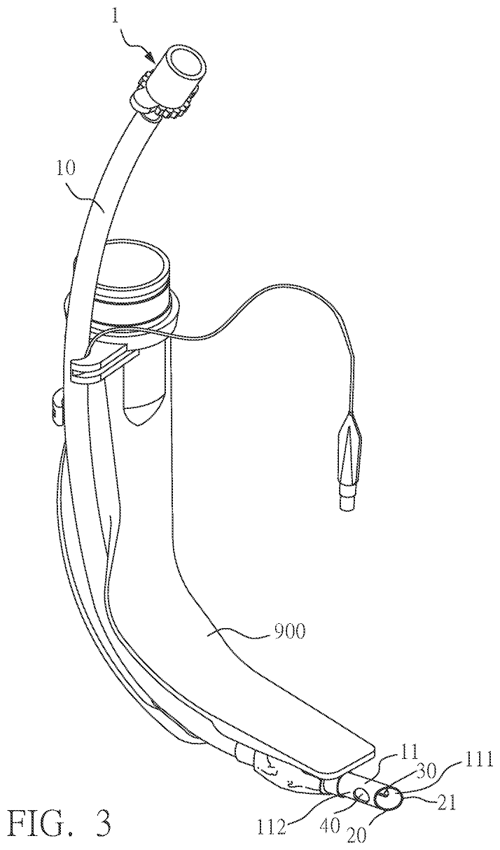

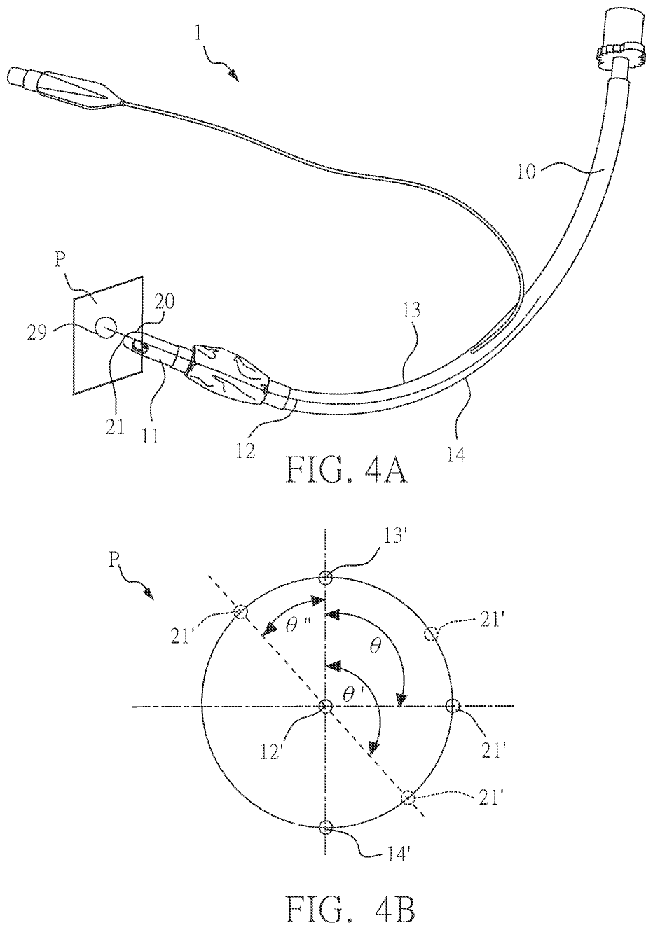

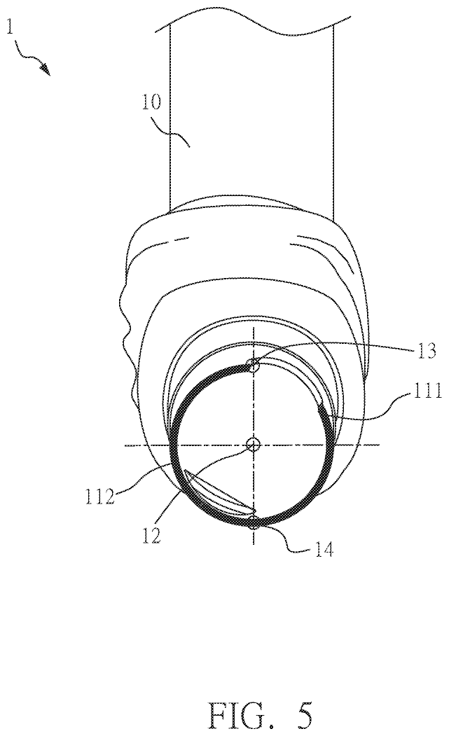

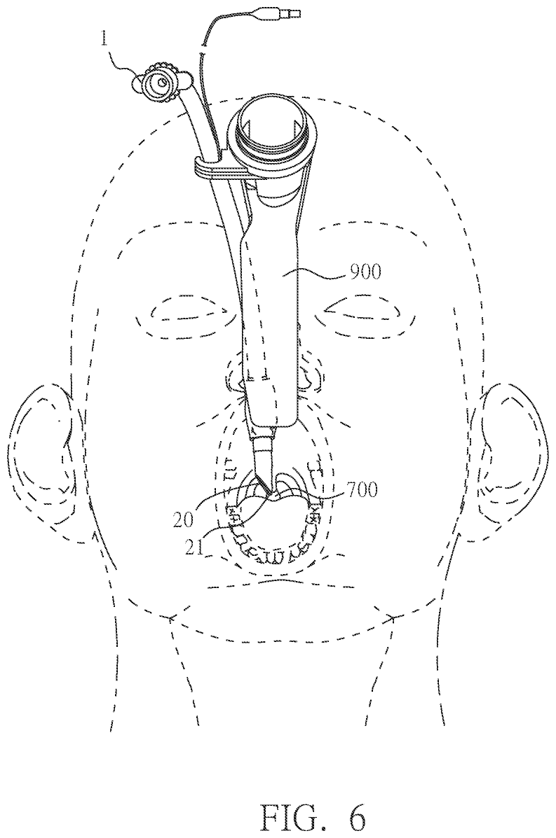

Please refer to FIG. 3 to FIG. 6, wherein FIG. 3 illustrates a schematic drawing of an endotracheal tube placed onto a laryngoscope blade according to a first embodiment of the present invention; FIG. 4A illustrates a schematic drawing of the endotracheal tube according to the first embodiment of the present invention; FIG. 4B illustrates a schematic drawing showing a circular projection surface on a vertical plane relative to an oblique cutting surface of the endotracheal tube according to the present invention; FIG. 5 illustrates a cross-sectional schematic drawing of a front end of the endotracheal tube according to the first embodiment of the present invention; and FIG. 6 illustrates a schematic drawing showing corresponding positions of the endotracheal tube placed onto the laryngoscope blade after being inserted into a human trachea according to the present invention.

As shown in FIG. 3, FIG. 4A and FIG. 4B, in the first embodiment, an endotracheal tube 1 of the present invention comprises a curved tube body 10 and an oblique cutting surface 20. The curved tube body 10 has a front end 11. The curved tube body 10 has a center axis 12, an inner axis 13 and an outer axis 14. The oblique cutting surface 20 has a tip 21, and the oblique cutting surface 20 is located at the front end 11. The endotracheal tube 1 of the present invention is characterized in that: the oblique cutting surface 20 has a circular projection surface 29 on a vertical plane P, the tip 21 has a tip projection point 21′ on the circumference of the circular projection s face 29, the inner axis 13 has an inner axis projection point 13′ on the circumference of the circular projection surface 29, and the tip projection point 21′ and the inner axis projection point limn an angle θ, where −45°<θ<90°. By means of changing the direction of the tip 21 of the oblique cutting surface 20, as shown in FIG. 6, the tip 21 of the endotracheal tube 1 of the present invention may face toward the center of a patient's tracheal passage 700 if the width of the patient's larynx is smaller than 4 cm so as to increase the success rate of endotracheal intubation, especially by increasing the success rate of endotracheal intubation on those patients having a smaller oral cavity or narrower larynx, which increase the difficulty of endotracheal intubation, and also to reduce the risk of soft tissue injury caused by the tip 21. Please note that, as shown in FIG. 4B, on the circular projection surface 29, the inner axis projection point 13′ is used as a starting point for defining moving right (i.e., moving along the clockwise direction) from the inner axis projection point 13′ as being positive, and moving left (i.e., moving along the counterclockwise direction) from the inner axis projection point 13′ as being negative. As a result, −45° means the tip projection point 21′ is located to the left of the inner axis projection point 13′, which is illustrated as angle θ″ in FIG. 4B. Similarly, θ<90° means the tip projection point 21′ is located to the right of the inner axis projection point 13′, which is illustrated as angle θ′ in FIG. 4B. Please note that, as shown in FIG. 4A, the tip 21 of the endotracheal tube 1 of the first embodiment would have its tip projection point 21′ projected onto the circumference of the circular projection surface 29 at the angle of θ=90°; however, please note that the scope of the present invention is not limited to the above description. In fact, 0°<θ<90° may be the preferred embodiment of the present invention, but it is to be noted that −25°<θ<45°, 15°<θ<45°, 0°<θ<45° and 0°<θ<135° are all applicable embodiments.

Please refer to the center axis 12, the inner axis 13 and the outer axis 14 of the curved tube body 10 as shown in FIG. 4A and FIG. 5, wherein the center axis 12 is an extended line of the pipe center of the curved tube body 10, the inner axis 13 is an axis located at the side of the curved tube body 10 and has a higher curvature when the curved tube body 10 bends naturally, and the outer axis 14 is an axis located at the side of the curved tube body 10 and has a lower curvature when the curved tube body 10 bends naturally.

As shown in FIG. 3, FIG. 4A, FIG. 4, FIG. 5 and FIG. 6, the front end 11 comprises a right side tube portion 111. and a left side tube portion 112. According to the endotracheal tube 1 of the present invention, positions of the right side tube portion 111 and the left side tube portion 112 are defined based on the viewing angle of the cross-sectional schematic drawing of the front end 11 as shown in FIG. 5, wherein the right side of the cross-sectional schematic drawing is the right side tube portion 111, and the left side of the cross-sectional schematic drawing is the left side tube portion 112. The cutting direction of the oblique cutting surface 20 of the endotracheal tube 1 of the present invention is oblique from the right side tube portion 111 to the left side tube portion 112, which is different from the cutting direction of the conventional endotracheal tube 800, such that when the tip 21 of the endotracheal tube 1 of the present invention is used with the laryngoscope blade 900, the tip 21 of the endotracheal tube 1 of the present invention will be located at the center of the tracheal passage 700, as shown in FIG. 6, so as to increase the success rate of endotracheal intubation, as well as to effectively reduce the risk of soft tissue injury caused by the tip 21.

Please refer to FIG. 3. FIG. 4B and FIG. 5. The endotracheal tube 1 of the present invention comprises a first opening 30 and a second opening 40. The first opening 30 and the second opening 40 are both located on the front end 11 and are respectively located on two opposite sides of the tip 21. According to the arrangement of the first opening 30 and the second opening 40, oxygen may enter the patient's lung to maintain the patient's respiration if the oblique cutting surface 20 is blocked. In the first embodiment of the present invention, the first opening 30 is located at the right side tube portion 111 and the second opening 40 is located at the left side tube portion 112, wherein the first opening 30 is elliptical in shape and the second opening 40 is circular in shape. The first opening 30 has a first aperture 31. the second opening 40 has a second aperture 41, and the first aperture 31 is greater than the second aperture 41. According to one preferred embodiment of the present invention, the size of the second aperture 41 is, but is not limited to, 20% to 80% of the size of the first aperture 31.

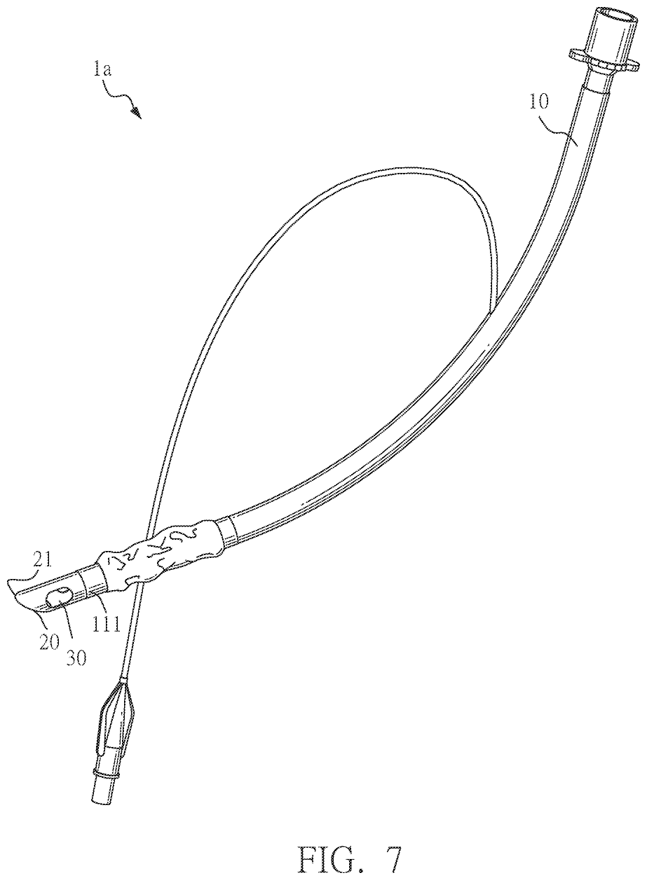

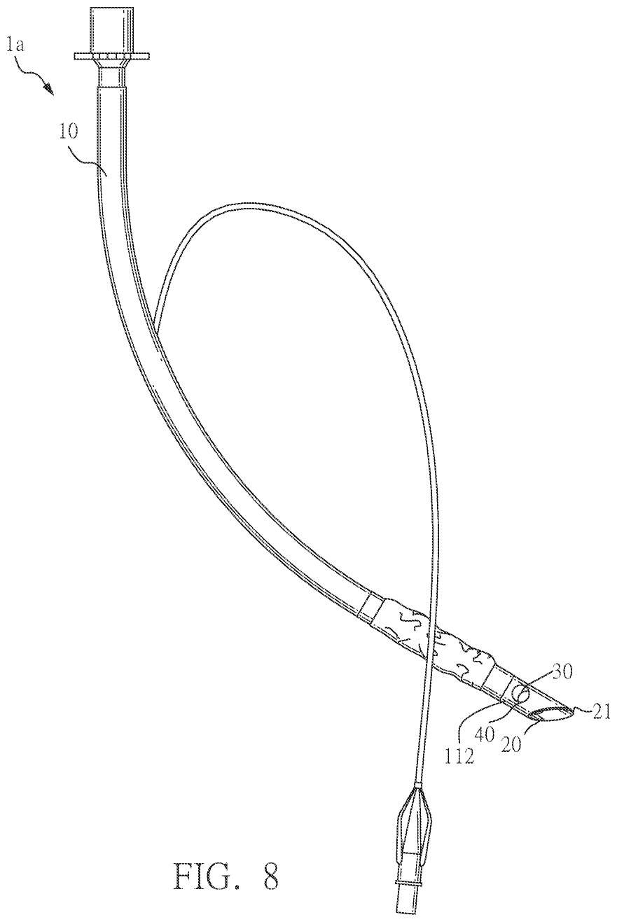

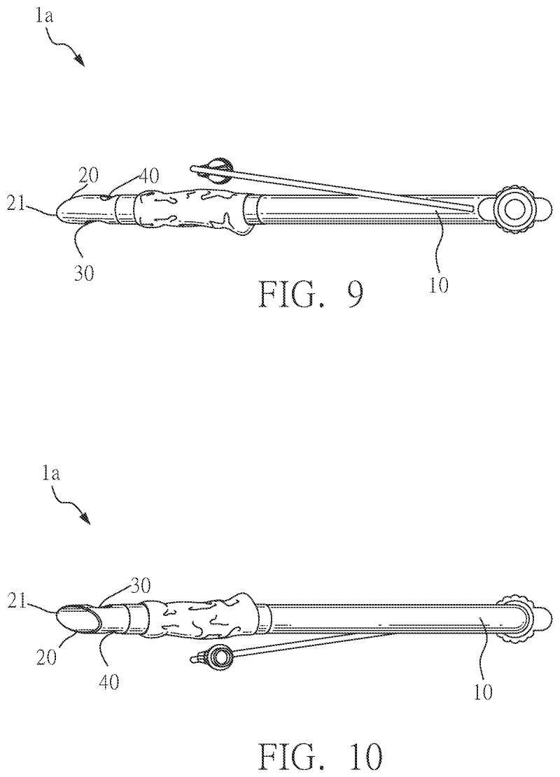

Please refer to FIG. 4B and FIGS. 7 to 10, which present the right side view, left side view, top view and bottom view of the endotracheal tube according to a second embodiment of the present invention.

As shown n FIG. 4B and FIGS. 7 to 10, the difference between the first embodiment and the second embodiment is that the tip 21 of the endotracheal tube 1a of the second embodiment has its tip projection point 21′ projected onto the circumference of the circular projection surface 29 at the angle of θ=0°. At this time, the first opening 30 and the second opening 40 are both located on the front end 11 and are respectively located on two opposite sides of the tip 21. The first opening 30 is located at the right side tube portion 111, and the second opening 40 is located at the left side tube portion 112.

The endotracheal tubes 1 and 1a of the present invention can make the tip 21 face toward the center of the patient's tracheal passage 700 upon movement of the laryngoscope blade 900 so as to increase the success rate of endotracheal intubation. Because the cutting direction of the oblique cutting surface 20 located at the front end 11 of the endotracheal tubes 1 and la of the present invention is oblique from the right side tube portion 111 to the left side tube portion 112, the tip 21 of the endotracheal tubes 1 and 1a can therefore face toward the center of the patient's tracheal passage 700 if the width of the patient's larynx is smaller than 4 cm so as to increase the success rate of endotracheal intubation, especially for those patients having a smaller oral cavity or narrower larynx, which increase the difficulty of endotracheal intubation, and also to reduce the risk of soft tissue injury caused by the tip 21.

Although the present invention has been explained in relation to its preferred embodiments, it is to be understood that many other possible modifications and variations can be made without departing from the spirit and scope of the invention as hereinafter claimed.

What is claimed is:

1. An endotracheal tube, comprising a curved tube body and an oblique cutting surface, the curved tube body having a front end and the curved tube body having a center axis, an inner axis and an outer axis, the oblique cutting surface having a tip and the oblique cutting surface being located at the front end, wherein the endotracheal tube is characterized in that: the oblique cutting surface has a circular projection surface on a vertical plane, the tip has a tip projection point on the circumference of the circular projection surface, the inner axis has an inner axis projection point on the circumference of the circular projection surface, and the tip projection point and the inner axis projection point form an angle θ, where 15°<θ<45°.

2. The endotracheal tube as claimed in claim 1, wherein −25°<θ<45°.

3. The endotracheal tube as claimed in claim 1, wherein 0°<θ<90°.

4. The endotracheal tube as claimed in claim 1, wherein 0°<θ<90°.

5. The endotracheal tube as claimed in claim 1, further comprising a first opening and a second opening, wherein the first opening and the second opening are both located on the front end and respectively located on two opposite sides of the tip.

6. The endotracheal tube as claimed in claim 5, wherein the first opening has a first aperture, the second opening has a second aperture, and the first aperture is greater in size than the second aperture.

7. The endotracheal tube as claimed in claim 6, wherein the size he second aperture is 20% to 80% of the size of the first aperture.

8. The endotracheal tube as claimed in claim 7, wherein the first opening is elliptical in shape and the second opening is circular in shape.

9. The endotracheal tube as claimed in claim 5, wherein the front end further comprises a right side tube portion and a left side tube portion, the first opening being located on the right side tube portion and the second opening being located on the left side tube portion.

10. The endotracheal tube as claimed in claim 9, wherein the first opening has a first aperture, the second opening has a second aperture, and the first aperture is greater in size than the second aperture.

11. The endotracheal tube as claimed in claim 10, wherein the size of the second aperture is 20% to 80% of the size of the first aperture.

12. The endotracheal tube as claimed in claim 11, wherein the first opening is elliptical in shape and the second opening is circular in shape.

13. The endotracheal tube as claimed in claim 1, wherein the endotracheal tube comprises a first opening) and a second opening, and the front end comprises a right side tube portion and a left side tube portion, the first opening and the tip being located on the right side tube portion and the second opening being located on the left side tube portion.

14. The endotracheal tube as claimed in claim 13, wherein the first opening has a first aperture, the second opening has a second aperture, and the first aperture is greater in size than the second aperture.

15. The endotracheal tube as claimed in claim 1.4, wherein the size of the second aperture is 20% to 80% of the size of the first aperture.

16. The endotracheal tube as claimed in claim 15, wherein the first opening is elliptical in shape and the second opening is circular in shape.
